(19) United States
(12) Patent Application Publication
(10) Pub. No.: US 2018/0078521 A1
(43) Pub. Date: Mar. 22, 2018

(54) COMPOSITIONS COMPRISING CAROTENOIDS AND USE THEREOF

(71) Applicant: ALGATECHNOLOGIES LTD., Kibbutz Ketura (IL)

(72) Inventors: Omer GRUNDMAN, Midreshet Ben-Gurion (IL); Hadas RICHTER, Beit-Elazari (IL); Santiago INI, Haifa (IL)

(21) Appl. No.: 15/564,162

(22) PCT Filed: Apr. 13, 2016
(86) PCT No.: PCT/IL2016/050389

§ 371 Date: Oct. 3, 2017

Related U.S. Application Data
(60) Provisional application No. 62/146,469, filed on Apr. 13, 2015.
(60) Provisional application No. 62/292,421, filed on Feb. 8, 2016.

(51) Int. Cl. A61K 31/336; A61K 36/03; A61K 31/20; A61K 31/201; A61K 31/202

(52) U.S. Cl. CPC A61K 31/336; A61K 36/03; A61K 31/202; A61K 31/201; A61K 31/20

(57) ABSTRACT

Microalgae extract and microalgae dried biomass compositions comprising carotenoids including but not limited to fucoxanthin and fatty acids, are provided.

### CROSS-REFERENCE TO RELATED APPLICATIONS

This application claims the benefit of U.S. Provisional Patent Application No. 62/146,469 filed Apr. 13, 2015 and U.S. Provisional Patent Application No. 62/292,421 filed Feb. 8, 2016, the contents of which are incorporated herein by reference in their entirety

### FIELD OF INVENTION

The present invention is directed to microalgae extract and microalgae dried biomass compositions including but not limited to, extracts comprising carotenoids and/or fatty acids.

### BACKGROUND OF THE INVENTION

Microalgae grow in either marine or freshwater systems. They are unicellular species which exist individually, or in chains or groups. Microalgae are capable of performing photosynthesis, and are primary producers in the oceans that convert water and carbon dioxide to biomass and oxygen. Microalgae species produce unique products such as carotenoids, antioxidants, fatty acids, enzymes, polymers, peptide, toxins and sterols.

Diatoms are microalgae, composed of a cell wall made primarily of silica and are mainly photosynthetic. Major pigments of diatoms are chlorophylls a and c, beta-carotene, fucoxanthin, diatoxanthin and diadinoxanthin.

Therapeutic supplements from micro-algae comprise an important market in which compounds such as β-carotene, astaxanthin, polyunsaturated fatty acid (PUFA) such as docosahexaenoic acid (DHA) and eicosapentaenic acid (EPA,) and polysaccharides such as β-glucan dominate.

Fucoxanthin is a carotenoid that exhibits anticancer, antioxidant, anti-diabetic, anti-obesity and anti-inflammatory properties. There is a need for microalgae biomass comprising high levels of fucoxanthin, alone or combined with additional carotenoids and/or fatty acids of nutraceutical value.

Further, extraction of fucoxanthin possess a challenge since it is unstable with respect to pH, temperature and light. In addition, when extracted from macro-algae the resulting extract contain heavy metals and iodine, obtained by the macro-algae. Therefore, there is a need for improved extracts comprising substantial amount of fucoxanthin which are essentially free of heavy metals and iodine and have low levels of saccharides.

### SUMMARY OF THE INVENTION

The present invention provides microalgae extract compositions exhibiting high levels of carotenoids, specifically fucoxanthin, and essential fatty acids together with low saccharide levels.

According to one aspect, there is provided a composition comprising a microalgae extract comprising: fucoxanthin and fatty acids, wherein monosaccharides and disaccharides constitute less than 0.7% by dry weight of the microalgae extract. In one embodiment, the extract comprises glucose, said glucose constitutes less than 0.1% by dry weight of the microalgae extract. In one embodiment, a ratio between the fucoxanthin and the monosaccharides and disaccharides is at least 4:1. In one embodiment, the fucoxanthin and the fatty acids constitute more than 2% and more than 30% by dry weight of said microalgae extract, respectively.

In another embodiment, the microalgae extract further comprises one or more carotenoids selected from diadinoxanthin, diatoxanthin and β-carotene, or isomers thereof.

In another embodiment, said fatty acids are selected from the group consisting of: saturated fatty acids, mono-unsaturated fatty acids, poly-unsaturated fatty acids, trans fatty acids or any combinations thereof. In another embodiment, said saturated fatty acids are one or more fatty acids selected from the group consisting of: butyric acid, caproic acid, capric acid, lauric acid, myristic acid, pentadecenoic acids, heptadecenoic acid, stearic acid, behenic acid, lignoceric acid, or isomers thereof. In another embodiment, said mono-unsaturated fatty acids are one or more fatty acids selected from the group consisting of: myristoleic acid, palmitoleic acid, oleic acid, docosenic acid, or isomers thereof. In another embodiment, said poly-unsaturated fatty acids are one or more fatty acids selected from the group consisting of: eicosapentaenic acid (EPA), linoleic acid, alpha linolenic acid, gamma linolenic acid, docosapentaenic acid, docosahexaenic acid (DHA), or isomers thereof.

According to another aspect, there is provided a composition comprising microalgae extract comprising: fucoxanthin, one or more carotenoids selected from diadinoxanthin, diatoxanthin and β-carotene or isomers thereof, palmitoleic acid, eicosapentaenic acid (EPA), archidonic acid, and docosahexaenic acid (DHA) or isomers thereof.

In another embodiment, said fucoxanthin constitutes more than 2% by dry weight of said microalgae extract. In another embodiment, said fucoxanthin constitutes more than 3% by dry weight of said microalgae extract. In another embodiment, said fucoxanthin constitutes more than 9% by dry weight of said microalgae extract.

In another embodiment, said palmitoleic acid constitutes more than 18% by dry weight of said microalgae extract. In another embodiment, said eicosapentaenic acid constitutes more than 20% by dry weight of said microalgae extract. In another embodiment, said archidonic acid constitutes more than 1% by dry weight of said microalgae extract. archidonic acid, said DHA constitutes more than 0.2% by dry weight of said microalgae extract.

In another embodiment, iodine constitutes less than 0.2 ppm by dry weight of said microalgae extract. In another embodiment, heavy metals constitutes less than 10 ppm by dry weight of the microalgae extract.

In another embodiment, said microalgae extract is obtained from microalgae selected from the group consisting of: Phaeodactylum tricornutum, Navicula pelliculosa, Amphora, Isochrysis aff. Galbana, Odontella aurita, Nitzscia closterium, Cylindrotheca closterium, Chaetoseros sp., and Emiliania huxleyi or a combination thereof.

According to another aspect, there is provided a composition comprising microalgae dried biomass comprising more than 1.6% fucoxanthin by dry weight. In another embodiment, monosaccharides and disaccharides constitute less than 2.7% by dry weight of the microalgae dried biomass.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1—High-Performance Liquid Chromatography with Diode-Array Detection (HPLC-DAD) chromatogram of P. tricornutum microalgae biomass recorded at 450 nm.

FIG. 2—HPLC-DAD chromatogram of P. tricornutum microalgae extract recorded at 450 nm.

### DETAILED DESCRIPTION OF THE INVENTION

In some embodiment, the invention provides microalgae extract compositions comprising high levels of one or more carotenoids and/or fatty acids. In some embodiment, the invention provides microalgae extract compositions comprising high levels of one or more carotenoids and/or fatty acids and low saccharide levels.

The present invention is based in part on the finding that the microalgae extracts of the invention has a unique composition which is advantageous for various fields and applications. As demonstrated hereinbelow, the microalgae extracts of the invention exhibit high fucoxanthin levels and extremely low saccharide levels.

In some embodiments, the microalgae extract or any formulation thereof may be used as a nutritional supplement, a pharmaceutical composition and/or cosmetic composition. For a non-limiting example, the microalgae extract may be incorporated in dry formulations of nutritional supplements and packaged in gel capsules, tablets, sachets and the like. In yet another example, the product may be useful in a liquid form for cosmetic preparations or packaging in soft capsules.

As used herein, the term “microalgae” means any unicellular, photosynthetic microorganism. In one embodiment, the microalgae extract is extracted from diatom microalgae. In one embodiment, the microalgae extract is extracted from P. tricornutum. In one embodiment, the microalgae extract is extracted from Navicula pelliculosa. In one embodiment, the microalgae extract is extracted from Amphora. In one embodiment, the microalgae extract is extracted from Isochrysis aff. Galbana. In one embodiment, the microalgae extract is extracted from Odontella aurita. In one embodiment, the microalgae extract is extracted from Nitzscia closterium. In one embodiment, the microalgae extract is extracted from Cylindrotheca closterium. In one embodiment, the microalgae extract is extracted from Chaetoseros sp. In one embodiment, the microalgae extract is extracted from Emiliania huxleyi.

In one embodiment, the microalgae is a wild type microalgae. In another embodiment, the microalgae is a genetically modified microalgae.

As used herein, the microalgae extract refers to materials extracted from microalgae. In one embodiment, microalgae can be harvested prior to extraction by any conventional means including, but not limited to filtration, air flotation and centrifugation.

### Extraction Methods

In one embodiment, the extraction is carried out by any means known in the art. In another embodiment, the extraction is a mechanical extraction. In another embodiment, the extraction is carried out by using an organic solvent. In one embodiment, the organic solvent is at least partially miscible in water. Non-limiting example of solvents that are miscible in water include methanol, ethanol, propanol, isopropanol, n-propanol, other alcohols containing 4 carbons or less, acetone, ketones containing 4 carbons or less, cyclic ethers such as dioxane and tetrahydrofuran, water miscible ethers such as diethyl ether, other oxygen-containing organic molecules having a ratio of carbon to oxygen atoms of about 4:1 or less and acetonitrile, or combination thereof. In another embodiment, the organic solvent is immiscible in water. Non-limiting examples of organic solvent that are immiscible in water include alkanes such as hexane, pentane, heptane, octane, esters such as ethyl acetate, butyl acetate, ketones such as methyl ethyl ketone (MEK), methyl isobutyl ketone (MIBK), aromatics such as toluene, benzene, cyclohexane, tetrahydrofuran, haloalkanes such as chloroform, trichloroethylene and ethers such as diethyl ether, or combinations thereof.

The term “polar solvent” as used herein means a solvent that tends to interact with other compounds or itself through acid-base interactions, hydrogen bonding, dipole-dipole interactions, or by dipole-induced dipole interactions. Non-limiting examples of polar solvents include: ethanol, propylene glycol, butylene glycol, methanol, glycerol, propanol, butanol, dipropylene glycol, pentylene glycol, hexylene glycol, dimethyl formamide, acetonitrile, dimethyl sulfoxide, dichloromethane, ethyl acetate, tetrahydrofuran, formic acid, acetic acid and acetone. Each possibility represents a separate embodiment of the invention. According to yet additional embodiments, the extraction is performed with a combination of at least two solvents.

In some embodiments, the carotenoid-containing microalgae extract, is in the form of an oleoresin, for example. The term “oleoresin” refers to a lipid extract of a carotenoid-containing material from microalgae.

In another embodiment, the extraction is carried out by using supercritical fluid-CO₂ (SCF-CO2) as known in the art. As used herein, supercritical fluid-CO₂ refer to CO₂ at a temperature (e.g., 40-60° C.) and pressure above its critical point, where distinct liquid and gas phases do not exist. In one embodiment, supercritical fluid-CO₂ can effuse through solids like a gas, and dissolve materials like a liquid. In another embodiment, the extraction is carried out by using SCF-CO₂ and a co-solvent. In one embodiment, the co-solvent is selected from ethanol, acetone, methanol, and any combination thereof.

In one embodiment, an extraction by a solvent is carried out following the SCF-CO2 extraction. In one embodiment, the extraction with a solvent is a liquid-liquid extraction. In one embodiment, the solvent is a polar solvent. In one embodiment, the solvent is selected from the group consisting of: ethanol, methanol, acetone, hexane and heptane. In some embodiment, the extraction by a solvent is followed by a second extraction by a second solvent. In some embodiments, the second solvent is a polar solvent.

The term “liquid-liquid extraction”, also known as solvent extraction and partitioning, refers to an extraction of a substance from one liquid into another liquid phase. In liquid-liquid extraction, substances are separated based on their relative solubilities in two different immiscible liquids (solvents), such as for a non-limiting example water and an organic solvent.

For a non-limiting example, the extraction is carried out by using supercritical fluid-CO₂ (SCF-CO2), followed by an extraction by a polar solvent, such as ethanol to enrich the ethanol extracted mass, which is followed by a second extraction with a second polar solvent (e.g., ethanol, ketone, ester, etc.).

### Microalgae Extract

In one embodiment, the microalgae extract comprises fucoxanthin in an amount of more than 1.7% or alternatively more than 1.8%, or alternatively more than 1.9%, or alternatively more than 2%, or alternatively more than 3%, or alternatively more than 4%, or alternatively more than 4%, or alternatively more than 5%, or alternatively more than 6%, or alternatively more than 7%, or alternatively more than 8%, or alternatively more than 9%, or alternatively more than 10%, or alternatively more than 11%, or alternatively more than 12%, or alternatively more than 13%, or alternatively more than 14%, by dry weight. Each possibility represents a separate embodiment of the present invention. In one embodiment, the microalgae extract comprises fucoxanthin in an amount of more than 2% by dry weight. In another embodiment, the microalgae extract comprises fucoxanthin in an amount of between 3% and 15% by dry weight.

As used herein, the term dry weight (DW) refers to the weight of the dry material.

In one embodiment, the microalgae extract comprises fucoxanthin and other carotenoids. In one embodiment, the microalgae extract comprises fucoxanthin and β-carotene or isomers thereof. In one embodiment, the microalgae extract comprises fucoxanthin and diadinoxanthin or isomers thereof. In one embodiment, the microalgae extract comprises fucoxanthin and diatoxanthin or isomers thereof.

In one embodiment, the microalgae extract further comprises fatty acids. In one embodiment, the fatty acids constitute more than 40%, or alternatively more than 45%, or alternatively more than 50%, or alternatively more than 55%, or alternatively more than 60%, or alternatively more than 70%, or alternatively more than 75%, or alternatively more than 80%, or alternatively more than 85% or alternatively more than 90% or alternatively more than 95% by dry weight of the microalgae extract. Each possibility represents a separate embodiment of the present invention.

In one embodiment, the fatty acids are selected from the group consisting of: saturated fatty acids, unsaturated fatty acids, trans fatty acids and any combinations thereof.

In one embodiment, the fatty acids are selected from the group consisting of: saturated fatty acids, mono-unsaturated fatty acids, poly-unsaturated fatty acids, trans fatty acids or any combinations thereof.

As demonstrated hereinbelow, a level of the saturated fatty acids in the microalgae extract is at least 5, 6, 7 or 8 folds lower than a level the saturated fatty acids in macro-algae extracts.

In one embodiment, the saturated fatty acids constitute more than 8%, or alternatively more than 9%, or alternatively more than 10%, or alternatively more than 11%, or alternatively more than 12%, or alternatively more than 13%, or alternatively more than 14%, or alternatively more than 15%, or alternatively more than 16% by dry weight of the microalgae extract. Each possibility represents a separate embodiment of the present invention. In one embodiment, the saturated fatty acids constitute less than 8%, or alternatively less than 9%, or alternatively less than 10%, or alternatively less than 11%, or alternatively less than 12%, or alternatively less than 13%, or alternatively less than 14%, or alternatively less than 15%, or alternatively less than 16%, or alternatively less than 20%, or alternatively less than 25% by dry weight of the microalgae extract. Each possibility represents a separate embodiment of the present invention. In one embodiment, the saturated fatty acids constitute less than 10% by dry weight of the microalgae extract. In one embodiment, the saturated fatty acids constitute less than 15% by dry weight of the microalgae extract. In one embodiment, the saturated fatty acids constitute between 5-20%, or alternatively between 5-18%, or alternatively between 6-18%, or alternatively between 7-18%, or alternatively between 5-15%, or alternatively between 5-10% by dry weight of the microalgae extract. Each possibility represents a separate embodiment of the present invention.

In one embodiment, the unsaturated fatty acids constitute more than 30%, or alternatively more than 35%, or alternatively more than 40%, or alternatively more than 45%, or alternatively more than 46%, or alternatively more than 50%, or alternatively more than 54%, or alternatively more than 55%, or alternatively more than 56% by dry weight of the microalgae extract. Each possibility represents a separate embodiment of the present invention. In one embodiment, the unsaturated fatty acids constitute between 40-70%, or alternatively between 45-60%, or alternatively between 50-70%, or alternatively between 50-65%, or alternatively between 50-60%, or alternatively between 55-65% by dry weight of the microalgae extract. Each possibility represents a separate embodiment of the present invention.

In one embodiment, the poly-unsaturated fatty acids constitute more than 15%, or alternatively more than 16%, or alternatively more than 17%, or alternatively more than 18%, or alternatively more than 19%, or alternatively more than 20%, or alternatively more than 21%, or alternatively more than 22%, or alternatively more than 23%, or alternatively more than 24%, or alternatively more than 25%, or alternatively more than 26%, or alternatively more than 27%, or alternatively more than 28%, or alternatively more than 29%, or alternatively more than 30%, or alternatively more than 31%, or alternatively more than 32% by dry weight of the microalgae extract. Each possibility represents a separate embodiment of the present invention. In one embodiment, the poly-unsaturated fatty acids constitute more than 20% by dry weight of the microalgae extract. In one embodiment, the poly-unsaturated fatty acids constitute more than 25% by dry weight of the microalgae extract. In one embodiment, the poly-unsaturated fatty acids constitute between 15-50%, or alternatively between 15-40%, or alternatively between 20-40%, or alternatively between 25-40%, or alternatively between 20-35%, or alternatively between 25-35% by dry weight of the microalgae extract. Each possibility represents a separate embodiment of the present invention.

In one embodiment, the mono-unsaturated fatty acids constitute more than 10%, or alternatively more than 11%, or alternatively more than 12%, or alternatively more than 13%, or alternatively more than 14%, or alternatively more than 15%, or alternatively more than 16%, or alternatively more than 17%, or alternatively more than 18%, or alternatively more than 19%, or alternatively more than 20% by dry weight of the microalgae extract. Each possibility represents a separate embodiment of the present invention. In one embodiment, the mono-unsaturated fatty acids constitute more than 15% by dry weight of the microalgae extract. In one embodiment, the mono-unsaturated fatty acids constitute more than 18% by dry weight of the microalgae extract. In one embodiment, the mono-unsaturated fatty acids constitute between 10-30%, or alternatively between 12-30%, or alternatively between 15-30%, or alternatively between 10-28%, or alternatively between 10-25%, or alternatively between 15-28%, or alternatively between 15-25% by dry weight of the microalgae extract. Each possibility represents a separate embodiment of the present invention.

In one embodiment, the trans fatty acids constitute more than 3%, or alternatively more than 3.5%, or alternatively more than 4%, or alternatively more than 4.5%, or alternatively more than 5%, or alternatively more than 5.5%, or alternatively more than 6%, or alternatively more than 6.5% by dry weight of the microalgae extract. Each possibility represents a separate embodiment of the present invention. In one embodiment, the trans fatty acids constitute more than 5% by dry weight of the microalgae extract. In one embodiment, the trans fatty acids constitute more than 6% by dry weight of the microalgae extract. In one embodiment, the trans fatty acids constitute between 3-15%, or alternatively between 4-15%, or alternatively between 3-10%, or alternatively between 3-9%, or alternatively between 4-10%, or alternatively between 4-9%, or alternatively between 5-9% by dry weight of the microalgae extract. Each possibility represents a separate embodiment of the present invention.

In one embodiment, the saturated fatty acids are one or more fatty acids selected from the group consisting of: butyric acid, caproic acid, capric acid, lauric acid, myristic acid, pentadecenoic acids, palmitic acid (PA), heptadecenoic acid, stearic acid, behenic acid, lignoceric acid, or isomers thereof.

In one embodiment, the mono-unsaturated fatty acids are one or more fatty acids selected from the group consisting of: myristoleic acid, palmitoleic acid, oleic acid, docosenic acid, or isomers thereof.

In one embodiment, the poly-unsaturated fatty acids are one or more fatty acids selected from the group consisting of: eicosapentaenic acid (EPA), linoleic acid, alpha linolenic acid, gamma linolenic acid, docosapentaenic acid, docosahexaenic acid (DHA), or isomers thereof.

In one embodiment, the microalgae extract further comprises palmitoleic acid or isomers thereof, wherein the palmitoleic acid constitutes more than 5%, or alternatively more than 8%, or alternatively more than 10%, or alternatively more than 11%, or alternatively more than 12%, 30 or alternatively more than 13%, or alternatively more than 14%, or alternatively more than 15%, or alternatively more than 16%, or alternatively more than 17%, or alternatively more than 18%, or alternatively more than 19%, or alternatively more than 20%, or alternatively more than 21%, or alternatively more than 22% by dry weight of the microalgae extract. Each possibility represents a separate embodiment of the present invention.

In one embodiment, the microalgae extract further comprises eicosapentaenic acid (EPA) or isomers thereof, wherein said eicosapentaenic acid constitutes more than 1.5% or alternatively more than 2%, or alternatively more than 3%, or alternatively more than 4%, or alternatively more than 5%, or alternatively more than 6%, or alternatively more than 7%, or alternatively more than 7.5%, or alternatively more than 10%, or alternatively more than 11%, or alternatively more than 12%, or alternatively more than 13%, or alternatively more than 14%, or alternatively more than 15%, or alternatively more than 16%, or alternatively more than 17%, or alternatively more than 18%, or alternatively more than 19%, or alternatively more than 20%, or alternatively more than 21%, or alternatively more than 22%, or alternatively more than 23%, or alternatively more than 24%, or alternatively more than 25%, by dry weight of the microalgae extract. Each possibility represents a separate embodiment of the present invention.

In one embodiment, the microalgae extract further comprises archidonic acid (AA) or isomers thereof, wherein said AA constitute more than 0.1% or alternatively more than 0.2%, or alternatively more than 0.5%, or alternatively more than 0.6%, or alternatively more than 0.7%, or alternatively more than 0.9%, or alternatively more than 1%, or alternatively more than 1.5%, or alternatively more than 2%, or alternatively more than 2.5%, or alternatively more than 3% by dry weight of the microalgae extract. Each possibility represents a separate embodiment of the present invention.

In one embodiment, the microalgae extract further comprises docosahexaenic acid (DHA) or isomers thereof, wherein said DHA constitute more than 0.1% or alternatively more than 0.15%, or alternatively more than 0.2%, or alternatively more than 0.3% by dry weight of the microalgae extract. Each possibility represents a separate embodiment of the present invention.

In one embodiment, the microalgae extract further comprises palmitic acid (PA) or isomers thereof, wherein said PA constitute more than 5% or alternatively more than 6%, or alternatively more than 7%, or alternatively more than 8%, or alternatively more than 8.5% by dry weight of the microalgae extract. Each possibility represents a separate embodiment of the present invention.

In one embodiment, the microalgae extract comprises: fucoxanthin and fatty acids. In some embodiments, the weight to weight ratio of the fucoxanthin to the fatty acids in the extract ranges between 1:10 and 1:30. In some embodiments, the weight to weight ratio of the fucoxanthin to the fatty acids ranges between 1:10 and 1:20.

In some embodiments, the extract comprises fucoxanthin and unsaturated fatty acids. In some embodiments, the weight to weight ratio of the fucoxanthin to the unsaturated fatty acids in the extract ranges between 1:5 and 1:30, 1:5 and 1:20, 1:10 and 1:30 or 1:10 and 1:20. In some embodiments, the unsaturated fatty acids comprises monounsaturated fatty acids and polyunsaturated fatty acids.

In some embodiments, the weight to weight ratio of the fucoxanthin to the mono and poly unsaturated fatty acids in the extract ranges between 1:5 and 1:30, 1:5 and 1:20, 1:10 and 1:30 or 1:10 and 1:20. In some embodiments, the weight to weight ratio of the fucoxanthin to the poly-unsaturated fatty acids of the extract ranges between 1:3 and 1:30, 1:3 and 1:20, 1:3 to 1:15, 1:3 to 1:10, 1:4 and 1:30, 1:4 and 1:20, 1:4 to 1:15, 1:4 to 1:10, 1:5 and 1:30, 1:5 and 1:20, 1:5 to 1:15 or 1:5 and 1:10. In some embodiments, the weight to weight ratio of the fucoxanthin to the mono-unsaturated fatty acids of the extract ranges between 1:3 and 1:30, 1:3 and 1:20, 1:3 to 1:15, 1:3 to 1:10, 1:4 and 1:30, 1:4 and 1:20, 1:4 to 1:15, 1:4 to 1:10, 1:5 and 1:30, 1:5 and 1:20, 1:5 to 1:15 or 1:5 and 1:10.

In one embodiment, the invention provides a composition comprising microalgae extract comprising: fucoxanthin, palmitoleic acid, eicosapentaenic acid (EPA), archidonic acid (AA), gamma linolenic acid, docosahexaenic acid (DHA) and palmitic acid (PA) or isomers thereof.

In one embodiment, the invention provides a composition comprising microalgae extract comprising: fucoxanthin, one or more carotenoids selected from diadinoxanthin, diatoxanthin and β-carotene or isomers thereof, palmitoleic acid, eicosapentaenic acid (EPA), archidonic acid (AA), gamma linolenic acid, docosahexaenic acid (DHA) and palmitic acid (PA) or isomers thereof.

In one embodiment, the fucoxanthin constitutes more than 1%, or alternatively more than 1.5%, or alternatively more than 1.6%, or alternatively more than 1.7%, or alternatively more than 1.8%, or alternatively more than 1.9%, or alternatively more than 2%, or alternatively more than 3%, or alternatively more than 4%, or alternatively more than 5%, or alternatively more than 6%, or alternatively more than 7%, or alternatively more than 8%, or alternatively more than 9%, or alternatively more than 10%, or alternatively more than 11%, or alternatively more than 12%, 30 or alternatively more than 13%, or alternatively more than 14%, by dry weight of said microalgae extract. Each possibility represents a separate embodiment of the present invention.

In one embodiment, the palmitoleic acid and/or isomers thereof constitute more than 5%, or alternatively more than 8%, or alternatively more than 10%, or alternatively more than 11%, or alternatively more than 12%, or alternatively more than 13%, or alternatively more than 14%, or alternatively more than 15%, or alternatively more than 16%, or alternatively more than 17%, or alternatively more than 18%, or alternatively more than 19%, or alternatively more than 20%, or alternatively more than 21%, or alternatively more than 22% by dry weight of the microalgae extract. Each possibility represents a separate embodiment of the present invention.

In one embodiment, the microalgae extract comprises: the fucoxanthin and the palmitoleic acid and/or isomers thereof. In some embodiments, the weight to weight ratio of the fucoxanthin to the palmitoleic acid in the extract ranges between 2:1 and 1:10, 2:1 and 1:5, 2:1 and 1:2, 1:1 and 1:10, 1:1 and 1:5, 1:1 and 1:2, 1:2 and 1:10, or 1:2 and 1:5. Each possibility represents a separate embodiment of the present invention.

In one embodiment, the eicosapentaenic acid (EPA) and/or isomers thereof constitute more than 1.5% or alternatively more than 2%, or alternatively more than 3%, or alternatively more than 4%, or alternatively more than 5%, or alternatively more than 6%, or alternatively more than 7%, or alternatively more than 8%, or alternatively more than 10%, or alternatively more than 11%, or alternatively more than 12%, or alternatively more than 13%, or alternatively more than 14%, or alternatively more than 15%, or alternatively more than 16%, or alternatively more than 17%, or alternatively more than 18%, or alternatively more than 19%, or alternatively more than 20%, or alternatively more than 21%, or alternatively more than 22%, or alternatively more than 23%, or alternatively more than 24%, or alternatively more than 25% by dry weight of the microalgae extract. Each possibility represents a separate embodiment of the present invention.

In one embodiment, the microalgae extract comprises: the fucoxanthin and the EPA. In some embodiments, the weight to weight ratio of the fucoxanthin to the EPA in the extract ranges between 2:1 and 1:10, 2:1 and 1:5, 2:1 and 1:2, 1:1 and 1:10, 1:1 and 1:5, 1:1 and 1:2, 1:2 and 1:10, 1:2 and 1:8, 1:2 and 1:7, or 1:2 and 1:6. Each possibility represents a separate embodiment of the present invention. In some embodiments, the weight to weight ratio of the fucoxanthin to the EPA in the extract ranges between 1:2 and 1:8.

In one embodiment, the archidonic acid (AA) and/or isomers thereof constitute more than 0.1% or alternatively more than 0.2%, or alternatively more than 0.5%, or alternatively more than 0.6%, or alternatively more than 0.7%, or alternatively more than 0.9%, or alternatively more than 1%, or alternatively more than 1.5%, or alternatively more than 2%, or alternatively more than 2.5%, or alternatively more than 3% by dry weight of the microalgae extract. Each possibility represents a separate embodiment of the present invention.

In some embodiments, the extract comprises fucoxanthin and archidonic acid (AA) and/or isomers thereof. In some embodiments, the weight to weight ratio of the fucoxanthin to the AA in the extract ranges between 4:1 and 1:2, 3:1 and 1:2, 2:1 and 1:2, 1:1 and 1:2, 1.5:1 and 1:1.5, 4:1 and 1:1, 3:1 and 1:1, 2:1 and 1:1, or 1.5:1 and 1:1. Each possibility represents a separate embodiment of the present invention. In some embodiments, the weight to weight ratio of the fucoxanthin to the AA in the extract ranges between 2:1 and 1:1. In some embodiments, the weight to weight ratio of the fucoxanthin to the AA in the extract ranges between 1.5:1 and 1:1.

In one embodiment, DHA and/or isomers thereof constitute more than 0.1% or alternatively more than 0.15%, or alternatively more than 0.2%, or alternatively more than 0.24%, or alternatively more than 0.3% by dry weight of the microalgae extract. Each possibility represents a separate embodiment of the present invention.

In one embodiment, the microalgae extract comprises: fucoxanthin and DHA. In some 15 embodiments, the weight to weight ratio of the fucoxanthin to the DHA in the extract ranges between 10:1 and 1:1, 8:1 and 1:1, 7:1 and 1:1, 6:1 and 1:1, 5:1 and 1:1, 4:1 and 1:1, 10:1 and 2:1, 8:1 and 2:1, 7:1 and 2:1, 6:1 and 2:1, 5:1 and 2:1, 4:1 and 2:1, 10:1 and 3:1, 8:1 and 3:1, 7:1 and 3:1, 6:1 and 3:1, 5:1 and 3:1, or 4:1 and 3:1. Each possibility represents a separate embodiment of the present invention. In some embodiments, the weight to weight ratio of the fucoxanthin to the DHA in the extract ranges between 6:1 and 2:1. In some embodiments, the weight to weight ratio of the fucoxanthin to the DHA in the extract ranges between 5:1 and 3:1.

In one embodiment, the PA and/or isomers thereof constitute more than 5% or alternatively more than 6%, or alternatively more than 7%, or alternatively more than 8%, or alternatively more than 8.5% by dry weight of the microalgae extract. Each possibility represents a separate embodiment of the present invention.

In one embodiment, the microalgae extract comprises: fucoxanthin and PA. In some embodiments, the weight to weight ratio of the fucoxanthin to the PA in the extract ranges between 2:1 and 1:10, 2:1 and 1:8, 2:1 and 1:7, 2:1 and 1:6, 2:1 and 1:5, 2:1 and 1:4, 1:1 and 1:10, 1:1 and 1:8, 1:1 and 1:7, 1:1 and 1:6, 1:1 and 1:5, 1:1 and 1:4, 1:2 and 1:10, 1:2 and 1:8, 1:2 and 1:7, 1:2 and 1:6, 1:2 and 1:5, 1:2 and 1:4, 1:3 and 1:10, 1:3 and 1:8, 1:3 and 1:7, 1:3 and 1:6, or 1:3 and 1:5. Each possibility represents a separate embodiment of the present invention. In some embodiments, the weight to weight ratio of the fucoxanthin to the PA in the extract ranges between 1:3 and 1:5. In some embodiments, the weight to weight ratio of the fucoxanthin to the PA in the extract ranges between 1:2 and 1:6.

In one embodiment of the invention, iodine constitutes less than 0.2 parts per million (ppm) by dry weight of the microalgae extract. In one embodiment of the invention, iodine constitutes less than 0.5 parts per million (ppm) by dry weight of the microalgae extract.

In one embodiment of the invention, heavy metals (e.g., mercury, lead, cadmium, arsenic etc.) constitute less than 10 ppm or less than 5 ppm by dry weight of the microalgae extract.

In some embodiment of the invention, monosaccharides and disaccharides constitute less than 1%, 0.9%, 0.8%, 0.7%, 0.6%, 0.5%, 0.4%, 0.3%, 0.2%, 0.1%, 0.05% of the microalgae extract. Each possibility represents a separate embodiment of the present invention. In some embodiment of the invention, monosaccharides and disaccharides constitute less than 0.1% of the microalgae extract. In one embodiment, the microalgae extract is substantially free of monosaccharides and disaccharides. In some embodiments, a microalgae extract substantially free of monosaccharides and disaccharides comprises 0.7%, 0.6%, 0.5%, 0.4%, 0.3%, 0.2%, 0.1% or less monosaccharides and disaccharides by dry weight. Each possibility represents a separate embodiment of the present invention. In some embodiments, a microalgae extract substantially free of monosaccharides and disaccharides comprises 0.7% or less monosaccharides and disaccharides by dry weight. In some embodiments, a microalgae extract substantially free of monosaccharides and disaccharides comprises 0.1% or less monosaccharides and disaccharides by dry weight. In some embodiments, the weight to weight ratio of the fucoxanthin to the monosaccharides and disaccharides is at least 4:1, at least 5:1, at least 7:1, at least 10:1, or at least 20:1.

In some embodiment, glucose constitutes less than 1%, 0.9%, 0.8%, 0.7%, 0.6%, 0.5%, 0.4%, 0.3%, 0.2%, 0.1%, 0.05% of the microalgae extract. Each possibility represents a separate embodiment of the present invention. In some embodiment, glucose constitutes less than 0.1% of the microalgae extract. In one embodiment, the microalgae extract is substantially free of glucose. In some embodiments, a microalgae extract substantially free of glucose comprises 0.5%, 0.4%, 0.3%, 0.2%, 0.1% or less glucose by dry weight. Each possibility represents a separate embodiment of the present invention. In some embodiments, a microalgae extract substantially free of glucose comprises 0.1% or less glucose by dry weight.

In some embodiments, the weight to weight ratio of the fucoxanthin to the glucose is at least 5:1, at least 7:1, at least 10:1, at least 20:1, at least 30:1, at least 40:1, or at least 50:1. Each possibility represents a separate embodiment of the present invention. In some embodiments, the weight to weight ratio of fucoxanthin to glucose, in the extract, ranges between 10:1 and 100:1, 10:1 and 50:1, 10:1 and 40:1, 10:1 and 30:1, 20:1 and 100:1, 20:1 and 50:1, 20:1 and 40:1, or 20:1 and 30:1. Each possibility represents a separate embodiment of the present invention. In some embodiments, the weight to weight ratio of fucoxanthin to glucose, in the extract, ranges between 20:1 and 40:1. In some embodiments, the weight to weight ratio of fucoxanthin to glucose, in the extract, is at least 20:1.

In some embodiment, sugar constitutes less than 1%, 0.9%, 0.8%, 0.7%, 0.6%, 0.5%, 0.4%, 0.3%, 0.2%, 0.1%, 0.05% of the microalgae extract. Each possibility represents a separate embodiment of the present invention. In some embodiments, the weight to weight ratio of the fucoxanthin to the sugar ranges between 2:1 and 10:1, 2.5:1 and 10:1, 3:1 and 10:1, 4:1 and 10:1, or 5:1 and 10:1. In some embodiments, the weight to weight ratio of fucoxanthin to saccharides is at least 2:1, 2.5:1, 3:1, 4:1, 5:1, or 10:1.

In some embodiment, saccharides constitute less than 2%, 1.5%, 1%, 0.9%, 0.8%, 0.7%, 0.6%, 0.5%, 0.4%, 0.3%, 0.2%, 0.1%, 0.05% of the microalgae extract. Each possibility represents a separate embodiment of the present invention.

In some embodiments, the weight to weight ratio of fucoxanthin to saccharides, in the extract, ranges between 2:1 and 10:1, 2.5:1 and 10:1, 3:1 and 10:1, 4:1 and 10:1, or 5:1 and 10:1. In some embodiments, the weight to weight ratio of fucoxanthin to saccharides is at least 2:1, 2.5:1, 3:1, 4:1, 5:1, or 10:1.

The term “saccharide” refers to a carbohydrate which is a polyhydroxy aldehyde or ketone, or derivative thereof. As used herein, the term “saccharide” encompasses monosaccharides, disaccharides, oligosaccharides and polysaccharides, or derivatives thereof. Monosaccharides, or simple sugars, consist of a single polyhydroxy aldehyde or ketone unit. As used herein, the term “monosaccharide” refers to the basic unit of carbohydrates. Non-limiting examples of monosaccharides include: mannose, glucose (dextrose), fructose, galactose, xylose, and ribose. The term “glucose” refers to a monosaccharide having the chemical formula, C₆H₁₂O₆, which is also known as D-glucose or dextrose. As used herein, the term “disaccharide” refers to carbohydrates composed of two monosaccharides. Non-limiting examples of disaccharides include: sucrose, lactose and maltose. Oligosaccharides typically contain from 2 to 10 monosaccharide units joined in glycosidic linkage. Polysaccharides (glycans) typically contain more than 10 such units. The term “sugar” generally refers to mono-, di- or oligosaccharides.

In one embodiment, the invention provides a composition comprising microalgae extract comprising fucoxanthin, wherein the extract is substantially free of monosaccharides and disaccharides. In one embodiment, the invention provides a composition comprising microalgae extract comprising: fucoxanthin and fatty acids, wherein the extract is substantially free of monosaccharides and disaccharides. In one embodiment, the invention provides a composition comprising microalgae extract comprising: fucoxanthin and fatty acids, wherein monosaccharides and disaccharides constitute less than 0.1% by dry weight of the microalgae extract. In one embodiment, the invention provides a composition comprising microalgae extract comprising: fucoxanthin, palmitoleic acid, eicosapentaenic acid (EPA), archidonic acid (AA), gamma linolenic acid, docosahexaenic acid (DHA) and palmitic acid (PA) or isomers thereof, wherein the extract is substantially free of monosaccharides and disaccharides. In one embodiment, the invention provides a composition comprising microalgae extract comprising: fucoxanthin, one or more carotenoids selected from diadinoxanthin, diatoxanthin and β-carotene or isomers thereof, palmitoleic acid, eicosapentaenic acid (EPA), archidonic acid (AA), gamma linolenic acid, docosahexaenic acid (DHA) and palmitic acid (PA) or isomers thereof, wherein the extract is substantially free of monosaccharides and disaccharides.

### Microalgae Production

In one embodiment, the microalgae are grown in a defined culture medium. A suitable culture medium is any medium known in the art that support the viability and growth of the microalgae. In one embodiment, the culture medium comprises a nitrogen source selected from the group consisting: nitrate (NO₃), ammonium (NH₄⁺) and urea (CH₄N₂O) or a combination thereof. In one embodiment, the culture medium comprises urea. In one embodiment, the medium comprises less than about 0.5 gram/liter urea. In one embodiment, the medium comprises between 0.3 gram/liter to 0.8 gram/liter urea. In one embodiment, the medium comprises between 0.5 gram/liter to 1 gram/liter urea. In one embodiment, the medium comprises between 1 gram/liter to 2 gram/liter urea. In one embodiment, the medium comprises between 0.5 gram/liter to 3 gram/liter urea. In one embodiment, the microalgae use the urea as a nitrogen source. In one embodiment, the microalgae use the urea as a sole source of nitrogen.

In one embodiment, the culture medium comprises phosphate. In one embodiment, the medium comprises less than 0.1 gram/liter phosphate. In one embodiment, the medium comprises between 0.05 to 0.5 gram/liter phosphate. In one embodiment, the medium comprises between 0.5 to 2 gram/liter phosphate. In one embodiment, the medium comprises more than 2 gram/liter phosphate.

In one embodiment, the culture medium comprises a salt selected from the group consisting: sodium chloride (NaCl), Magnesium Sulfate (MgSO4), Magnesium Chloride (MgCl2), Calcium Chloride (CaCl2) or a combination thereof. In one embodiment, the medium comprises less than 5-30 gram/liter NaCl. In one embodiment, the medium comprises between 8 to 27 gram/liter NaCl. In one embodiment, the medium comprises between 1 to 5 gram/liter NaCl. In one embodiment, the medium comprises between 5 to 10 gram/liter NaCl. In one embodiment, the medium comprises less than 27 gram/liter NaCl.

In one embodiment, the medium is substantially free of silica. As used herein a medium substantially free of silica comprises less than 0.01 gram/liter silica, or alternatively less than 0.05 gram/liter silica, or alternatively less than 0.1 gram/liter silica, or alternatively less than 0.5 gram/liter silica.

### Microalgae Biomass

In an alternative aspect, there is provided microalgae biomass. The term “biomass” refers to any living or recently dead biological cellular material derived from microalgae. In one embodiment, the microalgae biomass is obtained from microalgae cell culture. In one embodiment, the microalgae biomass is a harvested biomass. In one embodiment, the microalgae biomass is a dried product of microalgae cells.

A person skilled in the art will appreciate that, the biomass may be harvested by any conventional means including, but not limited to filtration, air flotation and centrifugation. Additionally, dried biomass may be produced by various process known in the art. Non-limiting examples of drying techniques which are commonly used include: drum drying, rotary drying, freeze drying, solar drying, and spray drying.

As used herein, “Drum drying” refers to a method used for drying out microalgae into a film or paste using a large rotating drum that slowly applies heat. “Rotary drying” is much like drum drying except that an air pump is used to alter the pressure in order to evaporate water. “Freeze drying” refers to a dehydration process which works by freezing the subject material and then reducing the surrounding pressure and adding enough heat to allow the frozen water in the material to sublime directly from the solid phase to the gas phase. “Solar drying” refers to a method which uses glass and lenses to focus and trap heat from the sun. “Spray drying” refers to a method of producing a dry powder from a liquid or slurry by rapidly drying with a hot gas.

In some embodiments, one or more stabilizers are added to the biomass prior to obtaining a dried biomass in order to stabilize the Fucoxanthin content of the biomass. In some embodiments the stabilizers are antioxidants. In some embodiments the stabilizers are lipophilic antioxidants. Non-limiting examples of antioxidants include: vitamin C, Ascorbyl palmitate, vitamin E, and rosemary oil.

In some embodiments, stabilizers are added to the biomass, such that the stabilizer constitute between 0.1% and 5% by weight of the biomass prior to drying.

In some embodiments, the stabilizers are added following the extraction process. In some embodiments, stabilizers are added to the microalgae extract, such that the stabilizer constitute between 0.1% and 5% by weight of the microalgae extract. For a non-limiting example, Vitamin E and/or Ascorbyl palmitate, which are both lipophilic materials, may be added to the microalgae extract.

Cheol-Ho Pan et al. (Appl Biochem Biotechnol (2012) 166:1843-1855) disclosed 1.533% fucoxanthin by dry weight of P. tricornutum extract. Notably, this was achieved by cultivating the microalgae in 30 Liter plastic cylinders. Attempts to reach high fucoxanthin contents using biomass production techniques (e.g., using photobioreactors) resulted in substantially lower fucoxanthin contents. As such, Guil-Guerrero (Journal of Food Biochemisby 25, 2001, 57-76) reported microalgae biomass production reaching less than 0.45% carotenoids having about 50% fucoxanthin content.

Thus, the present invention provides in some embodiments, a composition comprising microalgae dried biomass comprising more than 0.5%, more than 0.6%, more than 0.7%, more than 0.8%, more than 0.9%, more than 1%, more than 1.1%, more than 1.2%, more than 1.3%, more than 1.5%, more than 1.6%, more than 1.7%, more than 1.8%, more than 1.9%, more than 2%, more than 2.1%, more than 2.2%, more than 2.3%, more than 2.4% or more than 2.5% fucoxanthin by dry weight, said microalgae is cultured in a photobioreactor. Each possibility represents a separate embodiment of the present invention.

As used herein, the term “photobioreactor” refers to a device or system used to support a biologically active environment for the mass (e.g., above 100 Liter) cultivation and/or production of microorganisms capable of performing photosynthesis, such as microalgae. The photobioreactor supplies a specifically controlled environment, allowing utilization of a light source (e.g., sun light) for autotrophic growth of the microorganisms. Autotrophic growth refers to the capability of an organism to synthesize its own food from inorganic substances, using light or chemical energy.

In one embodiment, the invention provides a composition comprising microalgae dried biomass comprising more than 1% fucoxanthin by dry weight. In one embodiment, the invention provides a composition comprising microalgae dried biomass comprising more than 1.1%, or alternatively more than 1.2%, or alternatively more than 1.3%, or alternatively more than 1.4%, or alternatively more than 1.5%, or alternatively more than 1.6%, or alternatively more than 1.7%, or alternatively more than 1.8%, or alternatively more than 1.9%, or alternatively more than 2% fucoxanthin by dry weight. Each possibility represents a separate embodiment of the present invention.

In one embodiment, fucoxanthin constitutes at least 1%, or alternatively at least 1.2%, at least 1.3%, at least 1.4%, at least 1.5%, at least 1.6%, at least 1.7%, at least 1.8%, at least 1.9%, 15 at least 2% by dry weight of the microalgae dried biomass. Each possibility represents a separate embodiment of the present invention.

In one embodiment, the microalgae dried biomass comprises fucoxanthin and other carotenoids. In one embodiment, the microalgae dried biomass comprises fucoxanthin and β-carotene or isomers thereof. In one embodiment, the microalgae dried biomass comprises fucoxanthin and diadinoxanthin or isomers thereof. In one embodiment, the microalgae dried biomass comprises fucoxanthin and diatoxanthin or isomers thereof.

In one embodiment, the microalgae dried biomass further comprises fatty acids.

In one embodiment, the fatty acids constitutes more than 5%, or alternatively more than 6%, or alternatively more than 7%, or alternatively more than 8%, or alternatively more than 9%, or alternatively more than 10%, or alternatively more than 11%, or alternatively more than 12%, or alternatively more than 13%, or alternatively more than 14% by dry weight of the microalgae dried biomass. Each possibility represents a separate embodiment of the present invention.

In one embodiment, the saturated fatty acids constitute more than 4%, or alternatively more than 5%, or alternatively more than 6%, or alternatively more than 7%, or alternatively more than 8%, or alternatively more than 9% by dry weight of the microalgae dried biomass. Each possibility represents a separate embodiment of the present invention. In one embodiment, the saturated fatty acids constitute less than 4%, or alternatively less than 5%, or alternatively more than 6%, or alternatively less than 7%, or alternatively less than 8%, or alternatively less than 9% by dry weight of the microalgae dried biomass. Each possibility represents a separate embodiment of the present invention. Each possibility represents a separate embodiment of the present invention. In one embodiment, the saturated fatty acids constitute between 2 and 10%, 3 and 10%, 4 and 10%, 5 and 10%, 2 and 8%, 3 and 8%, 2 and 6%, or 3 and 6% by dry weight of the microalgae dried biomass. Each possibility represents a separate embodiment of the present invention.

In one embodiment, the unsaturated fatty acids constitute more than 4%, or alternatively more than 5%, or alternatively more than 6%, or alternatively more than 7%, or alternatively more than 8%, or alternatively more than 9% by dry weight of the microalgae dried biomass. Each possibility represents a separate embodiment of the present invention. In one embodiment, the unsaturated fatty acids constitute between 4% and 20%, between 4% and 15%, between 4% and 10%, between 5% and 20%, between 5% and 15%, between 5% and 10%, between 6% and 20%, between 6% and 15%, between 6% and 10%, between 7% and 20%, between 7% and 15%, 15 or between 7% and 10 by dry weight of the microalgae dried biomass. Each possibility represents a separate embodiment of the present invention.

In one embodiment, the poly-unsaturated fatty acids constitute more than 1%, or alternatively more than 2%, or alternatively more than 3%, or alternatively more than 3.5%, or alternatively more than 4%, or alternatively more than 4.5% by dry weight of the microalgae dried biomass. Each possibility represents a separate embodiment of the present invention. In one embodiment, the poly-unsaturated fatty acids constitute between 1% and 10%, 1% and 7%, 1% and 6%, 1% and 5%, 2% and 10%, 2% and 7%, 2% and 6%, 2% and 5%, 3% and 10%, 3% and 7%, 3% and 6%, 3% and 5% by dry weight of the microalgae dried biomass. Each possibility represents a separate embodiment of the present invention. In one embodiment, the poly-unsaturated fatty acids constitute between 3% and 5% by dry weight of the microalgae dried biomass.

In one embodiment, the mono-unsaturated fatty acids constitute more than 0.5%, or alternatively more than 1%, or alternatively more than 1.5%, or alternatively more than 2%, or alternatively more than 3%, or alternatively more than 3.5%, or alternatively more than 4%, or alternatively more than 4.5% by dry weight of the microalgae dried biomass. Each possibility represents a separate embodiment of the present invention. In one embodiment, the mono-unsaturated fatty acids constitute between 1% and 10%, 1% and 7%, 1% and 6%, 1% and 5%, 2% and 10%, 2% and 7%, 2% and 6%, 2% and 5%, 3% and 10%, 3% and 7%, 3% and 6%, or 3% and 5% by dry weight of the microalgae dried biomass. Each possibility represents a separate embodiment of the present invention. In one embodiment, the mono-unsaturated fatty acids constitute between 3% and 5% by dry weight of the microalgae dried biomass.

In one embodiment, the trans fatty acids constitute more than 0.4%, or alternatively more than 0.5%, or alternatively more than 0.6%, or alternatively more than 0.7%, or alternatively more than 1%, or alternatively more than 1.5%, or alternatively more than 2%, or alternatively more than 2.5%, or alternatively more than 3% by dry weight of the microalgae dried biomass. Each possibility represents a separate embodiment of the present invention. In one embodiment, the trans fatty acids constitute between 0.4% and 3%, 0.4% and 2%, 0.4% and 1.5%, 0.4% and 1%, 0.5% and 3%, 0.5% and 2%, 0.5% and 1.5%, or 0.5% and 1% by dry weight of the microalgae dried biomass. Each possibility represents a separate embodiment of the present invention. In one embodiment, the trans fatty acids constitute between 0.5% and 1% by dry weight of the microalgae dried biomass.

In one embodiment, the microalgae dried biomass further comprises one or more 15 carotenoids selected from diadinoxanthin, diatoxanthin and β-carotene or isomers thereof.

In one embodiment, the microalgae dried biomass comprising fucoxanthin further comprises palmitoleic acid and/or isomers thereof. In one embodiment, the palmitoleic acid and/or isomers thereof constitute more than 1.5%, or alternatively more than 2%, or alternatively more than 2.5%, or alternatively more than 3% by dry weight of the microalgae dried biomass.

In one embodiment, the microalgae dried biomass comprising fucoxanthin further comprises eicosapentaenic acid and/or isomers thereof. In one embodiment, the eicosapentaenic acid and/or isomers thereof constitute more than 1% or alternatively more than 1.5%, or alternatively more than 2%, or alternatively more than 3%, or alternatively more than 3.5%, or alternatively more than 3.6%, or alternatively more than 3.7%, or alternatively more than 4% by dry weight of the microalgae dried biomass. Each possibility represents a separate embodiment of the present invention.

In one embodiment, the microalgae dried biomass comprising fucoxanthin further comprises archionic acid and/or isomers thereof. In one embodiment, the AA and/or isomers thereof constitute more than 0.1% or alternatively more than 0.01%, or alternatively more than 0.02%, or alternatively more than 0.03%, or alternatively more than 0.04%, or alternatively more than 0.05%, or alternatively at least 0.06 by dry weight of the microalgae dried biomass.

In one embodiment, the microalgae dried biomass comprising fucoxanthin further comprises archidonic acid (AA) and/or isomers thereof. In one embodiment, the archionic acid and/or isomers thereof constitute about 0.2%-0.5%, or alternatively more than 0.2%-0.4%, or alternatively about 0.3%, by dry weight of the microalgae dried biomass. Each possibility represents a separate embodiment of the present invention.

In one embodiment, the microalgae dried biomass comprising fucoxanthin further comprises DHA and/or isomers thereof. In one embodiment, DHA and/or isomers thereof constitute more than 0.05%, or alternatively more than 0.9%, or alternatively more than 0.10%, or alternatively more than 0.11%, or alternatively more than 0.12%, or alternatively more than 0.13%, or alternatively more than 0.14%, or alternatively more than 0.15%, or alternatively more than 0.16% by dry weight of the microalgae dried biomass. Each possibility represents a separate embodiment of the present invention.

In one embodiment, the microalgae dried biomass comprising fucoxanthin further comprises PA and/or isomers thereof. In one embodiment, the PA and/or isomers thereof constitute more than 1% or alternatively more than 1.1%, or alternatively more than 1.2%, or alternatively more than 1.3%, or alternatively more than 1.4%, or alternatively at least 1.5%, or alternatively at least 2%, or alternatively at least 3%, by dry weight of the microalgae dried biomass. Each possibility represents a separate embodiment of the present invention.

In another embodiment, the invention provides a composition comprising microalgae dried biomass comprising: fucoxanthin, one or more carotenoids selected from diadinoxanthin, diatoxanthin and β-carotene or isomers thereof, palmitoleic acid, eicosapentaenic acid (EPA), archidonic acid (AA), gamma linolenic acid, docosahexaenic acid (DHA) and palmitic acid (PA) or isomers thereof.

In one embodiment, the microalgae dried biomass comprises less than 5%, 4.5%, 4%, 3.5%, 3%, 2.9%, 2.8%, 2.7%, 2.6% monosaccharides and disaccharides. Each possibility represents a separate embodiment of the present invention.

In one embodiment, the microalgae dried biomass comprises less than 5%, 4.5%, 4%, 3.5%, 3%, 2.9%, 2.8%, 2.7%, 2.6% glucose. Each possibility represents a separate embodiment of the present invention. In one embodiment, the microalgae dried biomass comprises less than 2.7% glucose.

In one embodiment, the microalgae dried biomass comprises less than 5%, 4.5%, 4%, 3.5%, 3%, 2.9%, 2.8%, 2.7%, 2.6% sugars. Each possibility represents a separate embodiment of the present invention.

In one embodiment of the invention, iodine constitutes less than 0.3 ppm by dry weight of the microalgae dried biomass. In one embodiment of the invention, heavy metals (e.g., mercury, led, cadmium, arsenic, etc.) constitute less than 0.5 ppm by dry weight of the microalgae dried biomass.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Materials and Methods

### Microalgea Growth and Cultivation

Phaeodactylum microalgae were maintained in a defined artificial seawater medium which was developed from a growth medium (JONES, R. F., H. L. SPEER, AND W. KURY. 1963) used for the culture of the red alga Porphyridium cruentum. This modified salt-water medium, designated SW, contains per liter of H₂O: 27 gram (g) of NaCl, 6.6 g of MgSO₄-7H₂O, 5.6 g of MgCl₂*6H₂O, 1.5 g of CaCl₂-2H₂O, 1.0 g of KNO₃, 0.07 g of KH₂PO4, 0.04 g of NaHCO₃, 1 ml of “iron stock solution” (18.6 g of Na₂EDTA and 2.4 g of FeCl₃ 6 H₂O/liter, pH 7), and 1 ml of “microelements” stock solution (40 mg of ZnCl₂, 600 mg of H₃BO₃, 15 mg of CoCl₂-6H₂O, 40 mg of CuCl₂-2H₂O, 488 mg of MnCl₂A4H₂O, and 37 mg of (NH4)6MoO₂₄-4H₂O per liter).

Phaeodactylum microalgae were cultivated at 20° C. Air, supplemented with 2% C02, was bubbled to maintain the culture pH at 7.5±0.5. The culture was harvested upon reaching a minimum biomass of 3.5 gram/Liter.

### Determination of Fucoxanthin Content in Biomass and Oleoresin

70-80 mg of biomass samples, or 20-25 mg of 5% fucoxanthin oleoresin samples, was diluted with 20 ml of methanol by sonication for 5 min in a 25 ml volumetric flask. After sonication and cooling to ambient temperature, the volume was adjusted to the final volume (25 ml) with methanol. The solutions were filtered through a 0.22 μm PVDF syringe filter before analysis by HPLC (injected in triplicate). In addition Fucoxanthin reference standard (fucoxanthin, Lot: CDX-00006296-010 obtained from Chromadex, USA with standard purity of 98.9% (HPLC) solution was prepared by diluting with methanol at a concentration of 50 ppm. This solution was well mixed and filtered through a 0.22 μm PVDF syringe filter before analysis by HPLC (injected in triplicate). Fucoxanthin, fucoxanthin isomers, and other carotenoids were identified in the analysis based on the retention time of the compounds in the chromatograms and the corresponding absorbance spectrum.

### Example 1

### Effect of Nitrogen Source on Fucoxanthin Accumulation in P. tricornutum Microalgae

P. tricornutum microalgae were cultivated for 3 days, on day 3, nitrogen was added in the form of KNO₃ or urea (CH₄N₂O) alternatively. The content of fucoxanthin was determined by HPLC on three time points. Result show that when cells are grown in the presence of 0.5 g/liter urea the percent of fucoxanthin by dry weight of the biomass (also referred to as dry weight %/DW) is increased.

TABLE 1
Effect of nitrate on Fucoxanthin production

| | Fucoxanthin [%/DW] | | |
|---|---|---|---|
| | day 3 | day 6 | day 8 |
| No additional | 1.24 | 0.96 | 0.77 |
| nitrogen | | | |
| KNO3 | 1.25 | 1.3 | 1.26 |
| Urea | 1.29 | 1.54 | 1.6 |

### Example 2

### Effect of Salt Concentration on Fatty Acid Accumulation in P. tricornutum Microalgae

P. tricornutum microalgae were cultivated in the presence of different concentrations of sodium chloride (NaCl). Results show that when sodium chloride concentration in the medium was reduced from 27 g/liter to 9 g/liter the percentage of PA, AA, DPA, DHA and EPA from total fatty acid was increased (see table 2).

TABLE 2
Effect of sodium chloride on total fatty acids (TFA) accumulation

| | | NaCl concentration | |
|---|---|---|---|
| Fatty acid | Units | 9 gram/Liter | 27 gram/Liter |
| Palmitoleic acid | %/TFA | 19.4 | 17.2 |
| | %/DW | 2.5 | 2.6 |
| AA | %/TFA | 3.5 | 2.3 |
| | %/DW | 0.5 | 0.3 |
| EPA | %/TFA | 26 | 20.5 |
| | %/DW | 3.4 | 3.1 |
| DPA | %/TFA | 3.3 | 2.5 |
| | %/DW | 0.4 | 0.4 |
| DHA | %/TFA | 1.2 | 0.2 |
| | %/DW | 0.8 | 0.1 |
| TFA | %/DW | 13.1 | 15.2 |

### Example 3

### Biomass Content of P. tricornutum Microalgae

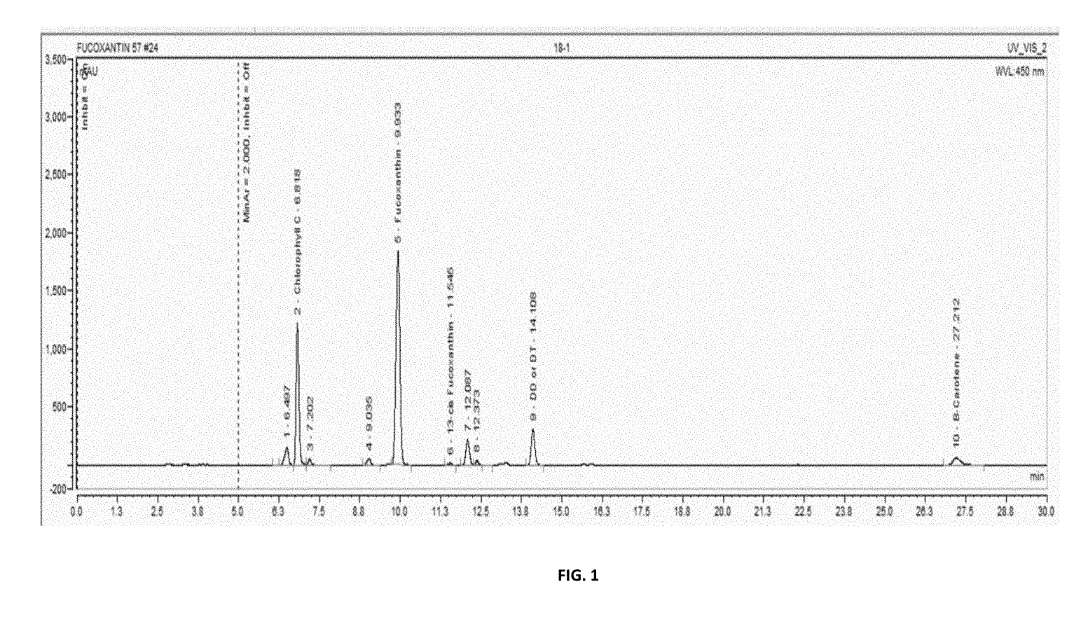

P. tricornutum microalgae were cultivated and harvested. The biomass content was examined by HPLC. A HPLC spectrum of the extract recorded at 450 nm is shown in FIG. 1.

The biomass content was analyzed and the calculated content in dry biomass is summarized in table 3.

TABLE 3
P. tricornutum microalgae biomass content

| | units | Value | Units | Value |
|---|---|---|---|---|
| Fuco | | | %/DW | 1.7-2 |
| Total Fatty Acids/DW | | | %/DW | 14.5 |
| C12:0 Laurie acid | %/total | 4-5 | %/DW | 0.03 |
| | fat | | | |
| C14:0 Myristic acid | %/total | 12.2 | %/DW | 1.04 |
| | fat | | | |
| C16:0 Palmitic acid | %/total | 0.2 | %/DW | 1.52 |
| | fat | | | |
| C16:1 Palmitoleic acid + | %/total | 7.2 | %/DW | 3.10 |
| isomeres | fat | | | |
| C16:3 Hexadecatrienoic acid | %/total | 0.3 | %/DW | 2.25 |
| (HTA) | fat | | | |
| C18:0 Stearic acid | %/total | 17.6 | %/DW | 0.03 |
| | fat | | | |
| C18:1-19 Oleic acid | %/total | 21.3 | %/DW | 0.06 |
| | fat | | | |
| C18:2cis/trans | %/total | 0.6 | %/DW | 0.01 |
| | fat | | | |
| C18:2 Linoleic acid | %/total | 7.3 | %/DW | 0.23 |
| | fat | | | |
| C18:3 Alpha Linolenic acid | %/total | 1.1 | %/DW | 0.10 |
| | fat | | | |
| C18:3 gamma-linolenic acid | %/total | 2.1 | %/DW | 0.04 |
| | fat | | | |
| C18:4 Octadecatetraenic acid | %/total | 0.1 | %/DW | 0.09 |
| | fat | | | |
| C20:0 Arachidic acid | %/total | 4.4 | %/DW | 0.06 |
| | fat | | | |
| C20:1 Eicosenoic acid + | %/total | 0.3 | %/DW | 0.03 |
| isomers | fat | | | |
| C20:2 Eicosodienoic acid + | %/total | 0.5 | %/DW | 0.04 |
| isomeres | fat | | | |
| C20:4 Arachidonic Acid | %/total | 0.6 | %/DW | 0.36 |
| | fat | | | |
| C20:5 Eicosapentaenic acid | %/total | 0.3 | %/DW | 4.04 |
| | fat | | | |
| C22:0 Behenic acid | %/total | 0.1 | %/DW | 0.03 |
| | fat | | | |
| C22:5 Docosapentaenic acid | %/total | 0.1 | %/DW | 0.19 |
| | fat | | | |
| C22:6 Docosahexaenic acid | %/total | 0.2 | %/DW | 0.16 |
| | fat | | | |
| C24:0 Lignoceric acid | %/total | 0.1 | %/DW | 0.62 |
| | fat | | | |
| C24:1 Tetracosenoic acid + | %/total | 3.1 | %/DW | 0.14 |
| isomeres | fat | | | |
| poly-unsaturated fatty acids | %/total | 25 | %/DW | 5.07 |
| | fat | | | |
| Others | %/total | 0.5 | %/DW | <0.1 |
| | fat | | | |

### Example 4

### Extract of P. tricornutum Microalgae

P. tricornutum microalgae were cultivated and harvested. The biomass was extracted by four alternative methods: ethanol extraction, SCF-CO2 extraction, SCF-CO2 and 2% ethanol extraction and SCF-CO2 followed by ethanol extraction (2 stages extraction). The contents of resulting extracts were compared to a control macro-algae (see table 4).

TABLE 4
Comparison of extracts content

| | | Ethanol | “2 stage” | | | SCF-CO2 + | Macro-algae |
|---|---|---|---|---|---|---|---|
| | Units | extract | extraction | SCF-CO2 | SCF-CO2 | 2% ethanol | extract |
| Fucoxanthin | %/DW | 4.4 | 6.3 | 6.2 | 6.4 | 8.8 | 5.5 |
| Purity | % | 69.0 | 61.8 | 69.6 | 72.9 | 72.6 | 85.66 |
| Total Fat | gr/100 gr | 52.4 | 81.0 | 72.5 | 70 | 79.5 | 91.4 |
| C8:0 Caprilyc | %/total | <0.1 | <0.1 | <0.1 | <0.1 | <0.1 | 52.7 |
| acid | fat | | | | | | |
| C10:0 Capric | %/total | <0.1 | <0.1 | <0.1 | <0.1 | <0.1 | 46.3 |
| acid | fat | | | | | | |
| C14:0 Myristic | %/total | 6 | 6.9 | 8.6 | 8.7 | 8.5 | <0.1 |
| acid | fat | | | | | | |
| C15:0 | %/total | 0.3 | 0.4 | 0.4 | 0.4 | 0.4 | <0.1 |
| Pentadecanic | fat | | | | | | |
| acid | | | | | | | |
| C16:0 Palmitic | %/total | 18.2 | 13.3 | 11.3 | 11.8 | 11.3 | 0.2 |
| acid | fat | | | | | | |
| C16:1 Palmitoleic | %/total | 22.7 | 27.9 | 24.6 | 25 | 24.3 | <0.1 |
| acid + isomers | fat | | | | | | |
| C18:0 Stearic | %/total | 0.7 | 0.7 | 0.3 | 0.3 | 0.3 | <0.1 |
| acid | fat | | | | | | |
| C18:1 trans | %/total | 8.4 | 8.0 | 8.2 | 7.9 | 8.4 | <0.1 |
| Elaidic acid | fat | | | | | | |
| C18:1-11 cis | %/total | 0.9 | 0.8 | 0.5 | 0.6 | 0.6 | <0.1 |
| Vaccenic acid | fat | | | | | | |
| C18:1-19 Oleic | %/total | 5.8 | 4.7 | 2 | 2.4 | 2.3 | 0.2 |
| acid | fat | | | | | | |
| C18:2cis/trans | %/total | 0.1 | 0.1 | <0.1 | <0.1 | 0.1 | <0.1 |
| | fat | | | | | | |
| C18:2 Linoleic | %/total | 6.7 | 8.2 | 3.9 | 4.1 | 4.2 | <0.1 |
| acid | fat | | | | | | |
| C18:2 trans/trans | %/total | 0.4 | 0.4 | 0.2 | 0.3 | 0.3 | <0.1 |
| | fat | | | | | | |
| C18:3 Alpha | %/total | 0.9 | 1.0 | 0.6 | 0.8 | 0.8 | 0.2 |
| Linolenic acid | fat | | | | | | |
| C18:3 gamma- | %/total | 0.6 | 0.6 | 0.5 | 0.5 | 0.6 | <0.1 |
| linolenic acid | fat | | | | | | |
| C18:4 | %/total | 0.4 | 0.3 | 0.4 | 0.4 | 0.4 | <0.1 |
| Octadecatetraenic | fat | | | | | | |
| acid | | | | | | | |
| C20:4 | %/total | 3.1 | 4.0 | 3.9 | 3.8 | 3.9 | <0.1 |
| Arachidonic Acid | fat | | | | | | |
| C20:5 | %/total | 22.4 | 20.4 | 32.8 | 31.5 | 32.3 | <0.1 |
| Eicosapentaenic | fat | | | | | | |
| acid | | | | | | | |
| C22:0 Behenic | %/total | 0.2 | 0.2 | <0.1 | <0.1 | <0.1 | <0.1 |
| acid | fat | | | | | | |
| C22:6 | %/total | 0.5 | 0.3 | 0.4 | 0.4 | 0.4 | <0.1 |
| Docosahexaenic | fat | | | | | | |
| acid | | | | | | | |
| C24:0 Lignoceric | %/total | 1 | 0.8 | 0.3 | 0.3 | 0.3 | <0.1 |
| acid | fat | | | | | | |
| C24:1 | %/total | 0.2 | 0.2 | 0.1 | 0.1 | 0.1 | <0.1 |
| Tetracosenoic | fat | | | | | | |
| acid + isomers | | | | | | | |
| Suturated Fatty | %/total | 26.5 | 22.4 | 21.1 | 21.7 | 21 | 99.4 |
| Acids total | fat | | | | | | |
| mono- | %/total | 29.7 | 33.9 | 27.5 | 28.2 | 27.5 | 0.2 |
| unsaturated fatty | fat | | | | | | |
| acids | | | | | | | |
| poly-unsaturated | %/total | 34.9 | 35.1 | 42.8 | 41.7 | 42.7 | 0.4 |
| fatty acids | fat | | | | | | |
| Total trans Fatty | %/total | 8.9 | 8.7 | 8.6 | 8.4 | 8.8 | <0.1 |
| acids | fat | | | | | | |
| Iodine | ppm | <0.2 | | | | | 0.9 |

### Example 5

### Fucoxanthin Content of P. tricornutum Microalgae Biomass and Extract

The content of fucoxanthin was determined in five samples of Phaeodactylum tricornutum. Fucoxanthin, its isomers and other carotenoids were quantified by HPLC. The analyzed samples include: Biomass sample and 10% fucoxanthin oleoresin: NX2677.

Fucoxanthin, fucoxanthin isomers, and other carotenoids were identified in the analysis based on the retention time of the compounds in the chromatograms and the corresponding absorbance spectrum.

All of the biomass samples of P. tricornutum presented fucoxanthin concentrations above 1% weight/weight (% w/w), as summarized in table 5.

Fucoxanthin minor isomer presented in the chromatogram is tentatively identified as 13-cis or 13′-cis. This affirmation is done on basis of retention times and UV-vis absorption spectra. According to scientific literature cis isomers of carotenoids show an additional λ peak about 330 nm (Crupi et al., 2013). This peak represents about 5% of total fucoxanthin in the sample, as summarized in table 6.

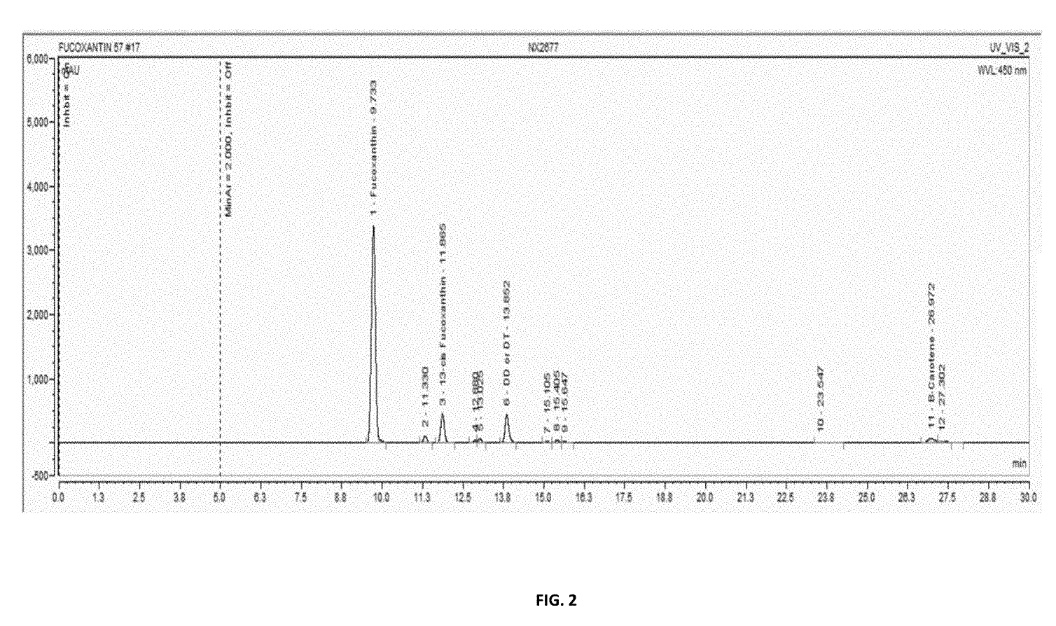

The presence of other carotenoids was also observed in the samples that have been identified as either diadinoxanthin or diatoxanthin and β-carotene (see FIGS. 1 and 2). This observation is sustained in the retention time, absorption spectra and scientific literature (Lavaud et al., 2002).

TABLE 5
Fucoxanthin content in P. tricornutum samples

| Sample | Compound | Result | unit |
|---|---|---|---|
| Biomass | Fucoxanthin | 1.31 ± 0.01 | %/DW |
| NX2677 | Fucoxanthin | 8.8 ± 0.5 | %/DW |
| Oleoresin | | | |

TABLE 6
Relative levels of fucoxanthin isomers in P. tricornutum
microalgae biomass

| Compound | Relative % |
|---|---|
| all trans fucoxanthin | 95.26% |
| 13 cis or 13′ cis fucoxanthin | 4.74% |
| total fucoxanthin | 100% |

### Example 6

### The Content of P. tricornutum Microalgae Biomass and Extract

P. tricornutum microalgae were cultivated and harvested. Table 7a summarizes the dry biomass content of the P. tricornutum microalgae, the content of oleoresin obtained from the P. tricornutum microalgae, and the content of oleoresin obtained from macro-algae.

As demonstrated in table 7a, an oleoresin obtained from P. tricornutum contains 19.06% eicosapentaenic acid (EPA), 2.38% archidonic acid (AA), and 13.4% palmitic acid (PA). Further, caprylic acid and capric acid constitute less than 0.02 and 0.05 of the content of the oleoresin obtained from P. tricornutum.

As further demonstrated in table 7a, the saturated fatty acids constitute 90.85% of the macro-algae extract and only 8.64% of the microalgae extract. fat content of an oleoresin obtained from macro-algae contains mostly caprilyc acid (48.17% from dry weight) and capric acid (42.32% from dry weight), wherein unsaturated fatty acids constitute only 0.55% of the dry weight.

Notably, as demonstrated in table 7b the glucose content in oleoresin obtained from P. tricornutum was under the detection limit of the measuring device that was used (presented in the table as less than 0.1). Further the content of mono and disaccharides was also undetectable (presented in the table as less than 0.7).

TABLE 7a
Biomass and oleoresin contents.

| | P. tricornutum | | Macro- |
|---|---|---|---|
| | Dry | | algae |
| | biomass | Oleoresin | Oleoresin |
| Fucoxanthin [%] | 1.5-2 | 3.12 | 5.52 |
| Total fat [%] | 11.53 | 67.34 | 91.4 |
| Caprylic acid in product [%] | <0.02 | <0.02 | 48.17 |
| Capric acid in product [%] | 0.01 | 0.05 | 42.32 |
| PA in product [%] | 3.26 | 13.40 | <0.1 |
| AA in product [%] | 0.35 | 2.38 | <0.1 |
| EPA in product [%] | 3.53 | 19.06 | <0.1 |
| DHA in product [%] | 0.21 | 0.78 | <0.1 |
| Total UFA in product [%] | 9.47 | 58.71 | 0.55 |
| Total PUFA in product [%] | 4.77 | 28.39 | 0.37 |
| Total MUFA IN product [%] | 4.24 | 30.32 | 0.18 |
| Total saturated FA in product [%] | 3.34 | 8.64 | 90.85 |

TABLE 7b
Biomass and oleoresin contents.

| | P. tricornutum | |
|---|---|---|
| | Dry biomass | Oleoresin |
| Fucoxanthin [%] | 1.5-2 | 3.12 |
| Protein [%] | 40.90 | 4.84 |
| Total fat [%] | 11.53 | 67.34 |
| Total UFA in product[%] | 9.47 | 58.71 |
| Total PUFA in product [%] | 4.77 | 28.39 |
| Glucose [%] | 2.63 | <0.1 |
| Sum of mono and disaccharides | 2.63 | <0.7 |
| Sodium [%] | 1.73 | 0.25 |

### Example 7

### Effect of Vitamin C and Rosemary Oil on Fucoxanthin Stability

P. tricornutum microalgae were cultivated and harvested. Vitamin C was added to the resulting biomass to constitute 1% by weight of the biomass. Alternatively, rosemary oil was added to the resulting biomass to constitute 0.3% by weight of the biomass. The percentage of Fucoxanthin was determined prior to drying the biomass, in the dry biomass and 7 days post drying of the biomass. Table 8 presents a comparison of Fucoxanthin content of a biomass treated with vitamin C, rosemary oil or for an untreated biomass. Results demonstrate that Fucoxanthin is stabilized when either Vitamin C or rosemary oil are added to the biomass. Notably, in the presence of both vitamin C as well as rosemary oil reduction in Fucoxanthin in time (see last column).

TABLE 8
Fucoxanthin stability under different condition

| | | | | % fucoxanthin | |
|---|---|---|---|---|---|
| | % fucoxanthin | % | % reduction | of the dried | % |
| | of the biomass | fucoxanthin | of fucoxanthin | biomass 7 days | reduction of |
| | prior to drying | of the dried | level due to the | post the drying | fucoxanthin |
| Treatment | of the biomass | biomass | drying process | process | level |
| No addition | 1.71 | 1.69 | 1.0% | 1.36 | 20.6% |
| Rosemary oil | 1.70 | 1.73 | −1.5% | 1.40 | 17.2% |
| Vitamin C | 1.72 | 1.77 | −3.0% | 1.52 | 11.1% |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A composition comprising a microalgae extract comprising fucoxanthin and fatty acids, wherein monosaccharides and disaccharides constitute less than 0.7% by dry weight of said microalgae extract.

2. The composition of claim 1, wherein said fucoxanthin and fatty acids constitute more than 2% and more than 30% by dry weight of said microalgae extract, respectively.

3. The composition of claim 1, wherein a ratio between said fucoxanthin and said monosaccharides and disaccharides is at least 4:1.

4. The composition of claim 1, wherein glucose constitutes less than 0.1% by dry weight of said microalgae extract.

5. The composition of claim 1, wherein said fucoxanthin constitutes more than 3% by dry weight of said microalgae extract.

6. The composition of claim 1, wherein said microalgae extract further comprises one or more carotenoids selected from diadinoxanthin, diatoxanthin and β-carotene, or isomers thereof.

7. The composition of claim 1, wherein said fatty acids are selected from the group consisting of: saturated fatty acids, mono-unsaturated fatty acids, poly-unsaturated fatty acids, trans fatty acids or any combinations thereof.

8. The composition of claim 7, wherein said saturated fatty acids are one or more fatty acids selected from the group consisting of: butyric acid, caproic acid, capric acid, lauric acid, myristic acid, pentadecenoic acids, heptadecenoic acid, stearic acid, behenic acid, lignoceric acid, or isomers thereof.

9. The composition of claim 7, wherein said mono-unsaturated fatty acids are one or more fatty acids selected from the group consisting of: myristoleic acid, palmitoleic acid, oleic acid, docosenic acid, or isomers thereof.

10. The composition of claim 7, wherein said poly-unsaturated fatty acids are one or more fatty acids selected from the group consisting of: eicosapentaenic acid (EPA), linoleic acid, alpha linolenic acid, gamma linolenic acid, docosapentaenic acid, docosahexaenic acid (DHA), or isomers thereof.

11. A composition comprising microalgae extract comprising: more than 2% fucoxanthin, one or more carotenoids selected from diadinoxanthin, diatoxanthin and β-carotene or isomers thereof, palmitoleic acid, eicosapentaenic acid (EPA), archidonic acid (AA), and docosahexaenic acid (DHA) or isomers thereof.

12. The composition of claim 11, wherein said fucoxanthin constitutes more than 3% by dry weight of said microalgae extract.

13. The composition of claim 11, wherein said fucoxanthin constitutes more than 9% by dry weight of said microalgae extract.

14. The composition of claim 11, wherein said palmitoleic acid constitutes more than 18% by dry weight of said microalgae extract.

15. The composition of claim 11, wherein any one of (i) said EPA constitutes more than 20% by dry weight of said microalgae extract; (ii) wherein said AA constitutes more than 0.3% by dry weight of said microalgae extract; (iii) said DHA constitutes more than 0.2% by dry weight of said microalgae extract; and (iv) any combination thereof.

16. (canceled)

17. (canceled)

18. The composition of claim 1, wherein iodine constitutes less than 0.2 ppm by dry weight of said microalgae extract.

19. The composition of claim 1, wherein heavy metals constitute less than 10 ppm by dry weight of said microalgae extract.

20. A composition comprising microalgae dried biomass comprising more than 1.6% fucoxanthin by dry weight.

21. The composition of claim 18, wherein monosaccharides and disaccharides constitute less than 2.7% by dry weight of said microalgae dried biomass.

22. The composition of claim 1, wherein said microalgae extract is obtained from microalgae selected from the group consisting of: Phaeodactylum tricornutum, Navicula pelliculosa, Amphora, Isochrysis aff. Galbana, Odontella aurita, Nitzscia closterium, Cylindrotheca closterium, Chaetoseros sp., and Emiliania huxleyi or a combination thereof.
